(19) United States
(12) Patent Application Publication
(10) Pub. No.: US 2024/0423285 A1
(43) Pub. Date: Dec. 26, 2024

(54) A CONSUMABLE FOR AN AEROSOL GENERATING APPARATUS

(71) Applicant: Imperial Tobacco Limited, Bristol (GB)

(72) Inventor: Matthew Pilkington, Liverpool Merseyside (GB)

(21) Appl. No.: 18/690,231

(22) PCT Filed: Sep. 7, 2022
(86) PCT No.: PCT/EP2022/074840

§ 371 Date: Mar. 7, 2024

(30) Foreign Application Priority Data
Sep. 7, 2021 (EP) 21195387.2

(51) Int. Cl. A24F 40/53; A24F 40/49; A24F 40/51; G06K 19/06

(52) U.S. Cl. CPC A24F 40/53; A24F 40/49; A24F 40/51; G06K 19/06093

(57) ABSTRACT

A consumable for forming an aerosol generating apparatus that comprises an aerosol forming precursor and data pertaining to the consumable encoded in machine-readable code. In some embodiments, the machine-readable code is optical code. Here, the machine readable optical code is provided on a surface of the consumable. The optical code suitably comprises a plurality of discrete code-portions provided at respective positions on said surface, and said data is distributed across said code-portions. By distributing the data across the plurality of discrete code-portions, the optical code can be read by a device and the data pertaining to the consumable stitched back together in software.

### FIELD

The present disclosure relates to the field of aerosol generating apparatuses formed by a consumer unit coupling with a consumable. In particular, the disclosure relates to a consumable, an aerosol generating apparatus comprising the consumable and the consumer unit, and a method of authenticating the consumable.

### BACKGROUND

Aerosol generating apparatuses may comprise a consumer unit that couples with a consumable assembly. The consumer unit is reusable and typically includes a power supply to activate a heater of the aerosol generating apparatus as well as general electrical circuitry for controlling an aerosol generation process. In use, a consumable assembly is operatively inserted or otherwise connected to the consumer unit and the consumer unit operated to generate an aerosol for inhalation by the user. The aerosol includes a suspension of precursor supplied by the consumable assembly. When the precursor in the consumable assembly has been consumed, the remainder of the consumable assembly is removed or disconnected from the consumer unit and discarded. In a subsequent operation, a second consumable assembly is inserted or otherwise connected to the consumer unit and a further aerosol generation process/processes completed. Manufacturers or suppliers brand their consumable assemblies to authenticate the consumable assembly as a genuine consumable assembly. A drawback is that the consumer unit is operable with consumable assemblies that replicate simple mechanical properties of the consumable assembly. Thus, the consumer unit generates an aerosol even when a non-genuine consumable assembly is knowingly or unknowingly inserted or otherwise connected to the consumer unit.

In spite of the effort already invested in the development of aerosol generating systems further improvements are desirable.

### SUMMARY

The present disclosure provides a consumable for forming an aerosol generating apparatus that comprises an aerosol forming precursor and data pertaining to the consumable encoded in machine-readable code.

In some embodiments, the machine-readable code is optical code. Here, the machine readable optical code is provided on a surface of the consumable. The optical code suitably comprises a plurality of discrete code-portions provided at respective positions on said surface, and said data is distributed across said code-portions. By distributing the data across the plurality of discrete code-portions, the optical code can be read by a device and the data pertaining to the consumable stitched back together in software. Distributing the information across a plurality of discrete code-portions provides further prevention against counterfeiting and can also require specific software to stitch the information back together. Thus, if the software includes a verification step, the consumable can provide an improved verification step. For instance, the software may be adapted to include a verification step such as a verification as to whether the consumable is a genuine consumable. Additionally or alternatively, the software may be adapted to include a verification step such as an age verification step. Additionally or alternatively, the software may be adapted to include a verification step such as a location verification step. It is envisaged in each verification step, the software compares information about the consumable and read from the machine readable-code when stitched back together, against reference information and verifies the consumable for use. Here, the software disables the aerosol generating apparatus from producing an aerosol if the consumable is not verified. Alternatively, the software may enable the aerosol generating apparatus to produce an aerosol if the consumable is verified.

In exemplary embodiments, the optical code is a QR code. Here, the QR code would be distributed across the plurality of discrete code-portions. Alternatively, each of the discrete code-portions forms a QR code. The or each QR code may comprise a Secure Quick Response Code (SQRC). In the exemplary embodiments, the optical code is suitably encrypted. For instance, the optical code includes a digital watermark or copy detection pattern to act to prevent reproduction of the optical code for use with counterfeit consumables.

According to the above exemplary embodiments there is therefore provided a consumable assembly that comprises an aerosol forming precursor and data pertaining to the consumable encoded as a distributed code across a plurality of discrete optical code-portions on a surface of the consumable assembly. There is also provided an aerosol generating apparatus comprising said consumable and a consumer device. When operatively assembled, the consumable and consumer unit are operable to produce an aerosol. There is also, further provided an aerosol generating system comprising said aerosol generating apparatus and an external device. For instance, the external device may be an external network or a mobile device such as a tablet or smart phone. In the exemplary embodiments, the consumer unit or the external device includes an optical detector. The optical detector being configured to read the optical code. For instance, the optical detector is a camera or other photo detector. The aerosol generating apparatus or aerosol generating system includes a processor for decoding the optical code and stitching the distributed information together to machine read said data pertaining to the consumable.

There is therefore also provided a method of reading the consumable having an aerosol forming precursor and data pertaining to the consumable encoded as a distributed code across a plurality of discrete optical code-portions on a surface of the consumable assembly. Here, the method comprises using an optical detector to read the plurality of discrete code-portions. The method further comprises processing the detected code-portions to stitch together data pertaining to the consumable that had been distributed across the code-portions.

In exemplary embodiments, the method of reading the consumable may include a verification step. For instance, an age verification, or a geographic location verification, or a genuine verification step. In the verification step, the data pertaining to the consumable is compared to a reference to complete the verification step. In embodiments including a distributed code, once the distributed code has been stitched back together, the verification step may be substantially as herein described. Here, in some embodiments, the aerosol generating apparatus or the aerosol generating system includes a processor. The processor is configured to complete the verification step. The processor may also be configured to enable or disable the aerosol generating process based on the outcome of the verification step. Thus, there is provided an aerosol generating apparatus, an aerosol generating system, and a method of producing an aerosol wherein a verification step enables or disables the aerosol generating process.

In some embodiments the machine readable code is read by a consumer unit or an external device. The data pertaining to the consumable is compared to a reference to complete a verification step. The consumer unit is configured to be enabled or disabled based on the verification step. Consequently, if the consumable, for instance, includes data pertaining to an age limit for use of the consumable, the reference can be a verified age of the user. If the verified age allows use of the consumable, the consumer unit can be enabled. Additionally, or alternatively, if the verified age does not allow use of the consumable, the consumer unit can be disabled. For instance, the consumer unit can be disabled until a further consumable is scanned with an allowable age limit. Additionally or alternatively, the consumable can include data pertaining to an allowable geographic location of use of the consumable. The reference can be a geographic location of the consumer unit or external device. Here the consumer unit can be enabled or disabled based on whether the reference location is permitted by the location data pertaining to the use of the consumable.

In some embodiments, the data pertaining to the consumable is data uniquely identifying the consumable. For instance, to uniquely identify the consumable from any other consumable. Here, the reference can be a reference to whether the consumable is authorised or authenticated. For instance, the data uniquely identifying the consumable can be looked up in a reference table and if the unique data is not present in the reference table, the consumable can be determined to be a counterfeit or non-authorised consumable. Here, the consumer unit can be disabled or not be enabled. If the unique data of the consumable is present in the reference table, the consumer unit can be enabled or not disabled. In addition, the reference table may include a further authentication data for the unique data. For instance, a manufacturer may be able to dynamically edit the reference for the unique data to validate or invalidate the consumable. Thus the consumer unit can be enabled or disabled even for genuine consumables. The reference for a unique consumable may be updated along the supply chain. For instance, the consumable can be tracked through transactions to the end user. Thus, if a genuine consumable becomes lost or stolen, the consumer unit can be disabled or not enabled upon scanning a lost or stolen genuine consumable. In some embodiments, the scanning of the consumable by the user device or external device to enable or not disable the consumer device can be updated in the reference for the unique data pertaining to the consumable and used to verify or not further scans of the same unique data.

According to the above exemplary embodiments there is therefore provided an aerosol generating apparatus comprising a consumer device and a consumable assembly that operatively engage to form an aerosol forming device. The consumable is able to be operatively coupled to the consumer device to form an aerosol generating apparatus. The consumable assembly includes the aerosol forming precursor and said data pertaining to the consumable encoded in machine-readable code. The machine readable code may be an optical code, for instance a QR code. Here, the QR code may be distributed across a plurality of code-portions. Alternatively to an optical code read by an optical sensor such as a camera, the machine readable-code may be another non-contact code such as an RFID tag read by an RFID reader, For instance, the consumable includes the RFID tag or passive element and the consumer deice comprises the RFID reader or interrogator. Here, the RFID reader or interrogator may also or alternatively provided on an external device that communicates with the consumer device in an aerosol generating system. In the exemplary embodiments, the consumer device includes a processor. The processor controls the operation of the consumer device. Here, the processor is configured to enable or disable the aerosol generation process of the consumer unit based on the result of a verification process. In the exemplary embodiments, the consumer unit communicates with an external device to determine a reference against which the data pertaining to the consumable and read from the machine readable code is compared as part of the verification step.

In the exemplary embodiments, the aerosol generating device includes an aerosol generating unit. Here, following the verification step, the aerosol generating unit can be enabled and/or disabled by a processor. For instance, the processor can control the power to the aerosol generation unit. In some exemplary embodiments, the aerosol generating system comprising the aerosol generating apparatus and an external device may be configured such that the aerosol generating apparatus is disabled unless it is operatively linked to an external device. For instance, unless the consumer unit of the aerosol generating apparatus is paired with an external device such as a tablet or mobile phone, the consumer unit is disabled. Here, as part of a communication interface, the tablet or external device can run an application to communicate with a communication module of the consumer unit. The application on the external device can include an age verification process. By arranging the consumer unit to verify each consumable, the system can be configured to complete the age verification process each time a new consumable is selected. By using a near field communication interface, the external device is required to be in near proximity to the consumer device when the consumable is scanned. Thus, making it harder for users to circumvent the age verification process.

According to the above, as well as an aerosol generating apparatus, there is provided an aerosol generation system comprising the aerosol generation apparatus and an external device. There is further provided a method of operating the aerosol generation system comprising reading a machine readable code contained on the consumable to determine data or information pertaining to the consumable. The determined data is compared against a reference. For instance, in exemplary embodiments, the reference is obtained from a communication interface that connects to a network. The reference is therefore recovered from a remote database. In the exemplary embodiments, the data pertaining to the consumable includes a unique reference to identify the consumable. The method may further comprise updating the remote reference to include information relating to the unique reference. For instance, the unique reference may be updated with information to identify transactions in the supply chain. Here, the verification process may include looking up the unique reference to determine whether the associated information permits a verification or not. For instance, if the unique reference has been updated with information determining the consumable has been lost or stolen, or that a previous step in the supply chain for the consumable has not been completed or completed properly, the processor can determine that the consumable is not verified. Thus, for instance, a shop retailer can scan the consumable and read the machine readable code. Here, the shop retailer updates the reference with information that the consumable has been sold correctly. Thus, when a user scans the consumable, the verification looks up the unique reference and determines the consumable is verified and allows or permits or does not disable the consumer device and the user can therefore operatively connect the consumable with the consumer unit and generate an aerosol. The aerosol generating system may be configured to update the reference to indicate that a consumer device has scanned the consumable. This indicator that the consumable has been scanned by a consumer unit may be used as a reference if the same unique consumable is scanned by a consumer unit in a subsequent operation.

In some exemplary embodiments, the processor is configured to complete the verification process each time a consumable is inserted or operatively coupled to the consumer unit. For instance, the consumer unit may include a sensor to detect when a consumable is removed. Here, the processor is configured to disable the aerosol generation process until a consumable is scanned.

In some exemplary embodiments, the machine readable code is an optical code. The optical code is provided on an outer surface of the consumable assembly. Where the consumable assembly is inserted or operatively engaged at one end of the consumable assembly, the optical code may be arranged on a part of the packaging at the other end. Thus the operatively connectable end of the consumable assembly does not include the optical code.

The present disclosure provides a use of the aerosol generating system, which may implement the features of any preceding embodiment, or another embodiment disclosed herein, for generating an aerosol for delivery to a user.

The present disclosure provides a method of generating an aerosol, which may implement the features of any preceding embodiment, or another embodiment disclosed herein. The method may include reading data pertaining to a consumable from a consumable. The data may be distributed across a plurality of discrete code-portions, in which case the method comprises stitching the code together in software to combine the code and read the data. The machine readable code may be optical code, in which case the data is read by an optical sensor. The method may comprise completing a verification step. The verification step compares the data pertaining to the consumable with a reference. Here, said compassion verifies the consumable or determines that the consumable cannot be verified. The method may comprise disabling an aerosol generation process if the consumable cannot be verified. Additionally or alternatively, the method may comprise enabling the aerosol generation process if the consumable is verified. The method may comprise operatively connecting the consumable with a consumer device to form an aerosol generating apparatus. The method may comprise operating the aerosol generating apparatus to generate an aerosol.

The present disclosure provides electrical circuitry, and/or a computer program configured to cause an aerosol generating apparatus or system to perform said method, and a computer readable medium comprising the computer program.

The preceding summary is provided for purposes of summarizing some embodiments to provide a basic understanding of aspects of the subject matter described herein. Accordingly, the above-described features are merely examples and should not be construed to narrow the scope or spirit of the subject matter described herein in anyway. Moreover, the above and/or proceeding embodiments may be combined in any suitable combination to provide further embodiments. Other features, aspects, and advantages of the subject matter described herein will become apparent from the following Detailed Description, Figures, and Claims.

### BRIEF DESCRIPTION OF THE FIGURES

Aspects, features and advantages of embodiments of the present disclosure will become apparent from the following description of embodiments in reference to the appended drawings in which like numerals denote like elements.

FIG. 1 is a block system diagram showing embodiment componentry of an aerosol generating apparatus.

FIG. 2 is a block system diagram showing embodiment componentry of the apparatus of FIG. 1.

FIG. 3 is a schematic diagram showing an embodiment of the apparatus of FIG. 2.

FIG. 4 is a block system diagram showing embodiment componentry of the apparatus of FIG. 1.

FIG. 5 is a schematic diagram showing an embodiment of the apparatus of FIG. 4.

FIG. 6 is a schematic diagram showing an aerosol generating system.

FIG. 7 is a schematic diagram showing an aerosol generating apparatus according to an exemplary embodiment.

FIG. 8 is a schematic diagram showing an aerosol generating system according to an exemplary embodiment.

FIG. 9 is a flow diagram of an exemplary method.

### DETAILED DESCRIPTION OF EMBODIMENTS

Before describing several embodiments of aerosol generating system and apparatus, it is to be understood that the system and apparatus is not limited to the details of construction or process steps set forth in the following description. It will be apparent to those skilled in the art having the benefit of the present disclosure that the systems, apparatuses and/or methods described herein could be embodied differently and/or be practiced or carried out in various ways.

Unless otherwise defined herein, scientific and technical terms used in connection with the presently disclosed inventive concept(s) shall have the meanings that are commonly understood by those of ordinary skill in the art, and known techniques and procedures may be performed according to conventional methods well known in the art and as described in various general and more specific references that may be cited and discussed in the present specification.

All of the systems, apparatus, and/or methods disclosed herein can be made and executed without undue experimentation in light of the present disclosure. While they have been described in terms of particular embodiments, it will be apparent to those of skill in the art that variations may be applied to the systems, apparatus, and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

The use of the term “a” or “an” in the present disclosure (including the claims) may mean “one,” as well as “one or more,” “at least one,” and “one or more than one.” As such, the terms “a,” “an,” and “the,” as well as all singular terms, include plural referents unless the context clearly indicates otherwise. Likewise, plural terms shall include the singular unless otherwise required by context.

The use of the term “or” in the present disclosure (including the claims) is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used in the present disclosure (including the claims), the words “comprising”, “having”, “including”, or “containing” (and any forms thereof, such as “comprise” and “comprises”, “have” and “has”, “includes” and “include”, or “contains” and “contain”, respectively) are inclusive or open-ended and do not exclude additional, unrecited elements or method steps.

Unless otherwise explicitly stated as incompatible, or the physics or otherwise of the embodiments, example, or claims prevent such a combination, the features of the foregoing embodiments and examples, and of the following claims may be integrated together in any suitable arrangement, especially ones where there is a beneficial effect in doing so. This is not limited to only any specified benefit, and instead may arise from an “ex post facto” benefit. This is to say that the combination of features is not limited by the described forms, particularly the form (e.g. numbering) of the example(s), embodiment(s), or dependency of the claim(s). Moreover, this also applies to the phrase “in one embodiment,” “according to an embodiment,” and the like, which are merely a stylistic form of wording and are not to be construed as limiting the following features to a separate embodiment to all other instances of the same or similar wording. This is to say, a reference to ‘an,’ ‘one,’ or ‘some’ embodiment(s) may be a reference to any one or more, and/or all embodiments, or combination(s) thereof, disclosed. Also, similarly, the reference to “the” embodiment may not be limited to the immediately preceding embodiment. Further, all references to one or more embodiments or examples are to be construed as non-limiting to the claims.

The present disclosure may be better understood in view of the following explanations, wherein the terms used that are separated by “or” may be used interchangeably:

As used herein, the term “aerosol generating apparatus” or “aerosol delivery apparatus” or “apparatus” or “electronic(e)-cigarette” may include apparatus to deliver an aerosol to a user for inhalation. The apparatus may also be referred to as a “smoking substitute apparatus”, which may refer to apparatus intended to be used instead of a conventional combustible smoking article. As used herein a “smoking article” may refer to a cigarette, cigar, pipe or other article, that produces smoke (an aerosol comprising solid particulates and gas) via heating above the thermal decomposition temperature (typically by combustion and/or pyrolysis). The apparatus may include an aerosol generating unit that may generate a vapour that may subsequently condense into the aerosol before delivery to an outlet, which may be arranged as a mouthpiece. The apparatus may be configured to deliver an aerosol for inhalation, which may comprise an aerosol with particle sizes of 0.2-7 microns, or less than 10 microns, or less than 7 microns. This particle size may be achieved by control of one or more of: heater temperature; cooling rate as the vapour condenses to an aerosol; flow properties including turbulence and velocity. The apparatus may be portable. As used herein, the term “Portable” may refer to the apparatus being for use when held by a user. The apparatus may be adapted to generate a variable amount of aerosol, e.g. by activating an aerosol generating unit of the apparatus for a variable amount of time, (as opposed to a metered dose of aerosol), which may be controlled by an input device. The input device may be configured to be user activated, and may for example include or take the form of a vaping button and/or inhalation sensor. Each occurrence of the aerosol generating apparatus being caused to generate aerosol for a period of time (which may be variable, see above) may be referred to as an “activation” of the aerosol generating apparatus. The aerosol generating apparatus may be arranged to vary an amount of aerosol delivered to a user based on the strength/duration of a draw of a user through a flow path of the apparatus (to replicate an effect of smoking a conventional combustible smoking article). As used herein, the term “aerosol generating system” or “aerosol delivery system” or “system” may include the apparatus and optionally other circuitry/componentry associated with the function of the apparatus, e.g. an external device and/or a external component (here “external” is intended to mean external to the aerosol generating apparatus). As used herein, the terms “external device” and “external component” may include one or more of: a mobile device (which may be connected to the aerosol generating apparatus, e.g. via a wireless or wired connection); a networked-based computer (e.g. a remote server); a cloud-based computer; any other server system.

As used herein, the term “aerosol” may include a suspension of precursor, including as one or more of: solid particles; liquid droplets; gas. Said suspension may be in a gas including air. Aerosol herein may generally refer to/include a vapour. Aerosol may include one or more components of the precursor.

As used herein, the term “aerosol-forming precursor” or “precursor” or “aerosol-forming substance” or “aerosol-forming substrate” may refer to one or more of a: liquid; solid; gel; loose leaf material; other substance. The precursor may be processable by an aerosol generating unit of the apparatus to form an aerosol. The precursor may include one or more of: an active component; a carrier; a flavouring. The active component may include one or more of nicotine; caffeine; a cannabidiol oil; a non-pharmaceutical formulation, e.g. a formulation which is not for treatment of a disease or physiological malfunction of the human body. The active component may be carried by the carrier, which may be a liquid, including propylene glycol and/or glycerine. The term “flavouring” may refer to a component that provides a taste and/or a smell to the user. The flavouring may include one or more of: Ethylvanillin (vanilla); menthol, Isoamyl acetate (banana oil); or other. The precursor may include a substrate, e.g. reconstituted tobacco to carry one or more of the active component; a carrier; a flavouring.

As used herein, the term “electrical circuitry” or “electric circuitry” or “circuitry” or “control circuitry” may refer to one or more of the suitable hardware or software components, examples of which may include: an Application Specific Integrated Circuit (ASIC); electronic/electrical componentry (which may include combinations of transistors, resistors, capacitors, inductors etc); one or more processors; a non-transitory memory (e.g. implemented by one or more memory devices), that may store one or more software or firmware programs; a combinational logic circuit; interconnection of the aforesaid. The electrical circuitry may be located entirely at the apparatus, or distributed between the apparatus and/or on one or more external devices in communication with the apparatus, e.g. as part of the system.

As used herein, the term “processor” or “processing resource” may refer to one or more units for processing, examples of which include an ASIC, microcontroller, FPGA, microprocessor, digital signal processor (DSP) capability, state machine or other suitable component. A processor may be configured to execute a computer program, e.g. which may take the form of machine readable instructions, which may be stored on a non-transitory memory and/or programmable logic. The processor may have various arrangements corresponding to those discussed for the circuitry, e.g. on-board and/or off board the apparatus as part of the system. As used herein, any machine executable instructions, or computer readable media, may be configured to cause a disclosed method to be carried out, e.g. by a aerosol generating device or system as disclosed herein, and may therefore be used synonymously with the term method, or each other.

As used herein, the term “external device” or “external electronic device” or “electronic user device” or “peripheral device” may include electronic components external to the apparatus, e.g. those arranged at the same location as the apparatus or those remote from the apparatus. The external electronic device may comprise electronic computer devices including: a smartphone; a PDA; a video game controller; a tablet; a laptop; or other like device.

As used herein, the term “computer readable medium/media” or “data storage” may include any medium capable of storing a computer program, and may take the form of any conventional non-transitory memory, for example one or more of: random access memory (RAM); a CD; a hard drive; a solid state drive; a memory card; a DVD. The memory may have various arrangements corresponding to those discussed for the circuitry/processor.

As used herein, the term “information carrying medium” may include one or more arrangements for storage of information on any suitable medium. Examples include: computer readable medium/media or data storage as defined herein; a Radio Frequency Identification (RFID) transponder; codes encoding information, such as optical (e.g. a bar code or QR code) or mechanically read codes (e.g. a configuration of the absence or presents of cut-outs to encode a bit, through which pins or a reader may be inserted).

As used herein, the term “communication resources” or “communication interface” may refer to hardware and/or firmware for electronic information transfer. The communication resources/interface may be configured for wired communication (“wired communication resources/interface”) or wireless communication (“wireless communication resources/interface”). Wireless communication resources may include hardware to transmit and receive signals by radio and may include various protocol implementations e.g. the 802.11 standard described in the Institute of Electronics Engineers (IEEE) and Bluetooth™ from the Bluetooth Special Interest Group of Kirkland Wash. Wired communication resources may include; Universal Serial Bus (USB); High-Definition Multimedia Interface (HDMI) or other protocol implementations. The apparatus may include communication resources for wired or wireless communication with an external device.

As used herein, the term “network” or “computer network” may refer to a system for electronic information transfer between a plurality of apparatuses/devices. The network may, for example, include one or more networks of any type, which may include: a Public Land Mobile Network (PLMN); a telephone network (e.g. a Public Switched Telephone Network (PSTN) and/or a wireless network); a local area network (LAN); a metropolitan area network (MAN); a wide area network (WAN); an Internet Protocol Multimedia Subsystem (IMS) network; a private network; the Internet; an intranet.

It will be appreciated that any of the disclosed methods (or corresponding apparatuses, programs, data carriers, etc.) may be carried out by either a host or client, depending on the specific implementation (i.e. the disclosed methods/apparatuses are a form of communication(s), and as such, may be carried out from either ‘point of view’, i.e. in corresponding to each other fashion). Furthermore, it will be understood that the terms “receiving” and “transmitting” encompass “inputting” and “outputting” and are not limited to an RF context of transmitting and receiving electromagnetic (e.g. radio) waves. Therefore, for example, a chip or other device or component for realizing embodiments could generate data for output to another chip, device or component, or have as an input data from another chip, device, or component, and such an output or input could be referred to as “transmit” and “receive” including gerund forms, that is, “transmitting” and “receiving,” as well as such “transmitting” and “receiving” within an RF context.

As used herein, the term “storage portion” may refer to a portion of the apparatus adapted to store the precursor, it may be implemented as fluid holding reservoir or carrier for solid material depending on the implementation of the precursor as defined above.

As used herein, the term “flow path” may refer to a path or enclosed passageway through the apparatus, through which the user may inhale for delivery of the aerosol. The flow path may be arranged to receive aerosol from an aerosol generating unit. When referring to the flow path, upstream and downstream may be defined in respect of a direction of flow in the flow path, e.g. the outlet is downstream of the inlet.

As used herein, the term “delivery system” may refer to a system operative to deliver an aerosol to a user. The delivery system may include a mouthpiece/a mouthpiece assembly and the flow path.

As used herein, the term “flow” may refer to a flow in the flow path. The flow may include aerosol generated from the precursor. The flow may include air, which may be induced into the flow path via a puff.

As used herein, the term “inhale” or “puff” or “draw” may refer to a user expansion of the lungs and/or oral cavity to create a pressure reduction that induces flow through the flow path.

As used herein, the term “aerosol generating unit” may refer to a device to form the aerosol from the precursor. The aerosol generating unit may include a unit to generate a vapour directly from the precursor (e.g. a heating system or other system) or an aerosol directly from the precursor (e.g. an atomiser including an ultrasonic system, a flow expansion system operative to carry droplets of the precursor in the flow without using electrical energy or other system). A plurality of aerosol generating units to generate a plurality of aerosols (for example, from a plurality of different aerosol precursors) may be present in the apparatus.

As used herein, the term “heating system” may refer to an arrangement of one or more heating elements, which are operable to aerosolise the precursor once heated. The heating elements may be electrically resistive to produce heat from electrical current therethrough. The heating elements may be arranged as susceptors to produce heat when penetrated by an alternating magnetic field. The heating system may heat the precursor to below 300 or 350 degrees C., including without combustion.

As used herein, the term “consumable” or “consumable assembly” may refer to a unit that includes or consists of the precursor. The consumable assembly may include the aerosol generating unit, e.g. it is arranged as a cartomizer. The consumable assembly may include the mouthpiece. The consumable assembly may include the information carrying medium. With liquid or gel implementations of the precursor, e.g. an E-liquid, the consumable assembly may be referred to as a “capsule” or a “pod” or “E-liquid consumable”. Here, the consumable assembly (e.g. capsule) may include the storage portion, e.g. a reservoir, for storage of the precursor. Typically for a capsule implementation, the consumable assembly comprises a housing that defines or otherwise comprises the storage portion. The storage portion may be refillable with liquid or gel or the consumable assembly may be configured to be disposable or recyclable one the liquid or gel has been consumed or otherwise depleted. As explained above, in a capsule implementation of the consumable assembly, the housing may optionally house or define the aerosol generating unit, mouthpiece, and information carrying medium. With solid material implementations of the precursor, e.g. tobacco or reconstituted tobacco formulation, the consumable assembly may be referred to as a “stick” or “package” or “heat not burn consumable”. When implemented as a heat not burn consumable, the consumable assembly may include a mouthpiece, for instance a mouthpiece that may be implemented as a filter. Further, in a heat not burn consumable assembly, a consumable material, being the tobacco or reconstituted tobacco formulation, may be arranged to carry the precursor. Here, the consumable assembly may include a wrapping material to wrap and store the precursor in the form of the consumable material. The consumable assembly may be implemented as a dosage or pre-portioned amount of material, including a loose-leaf product.

As used herein the term “consumer unit” or “consumer device” or “user device” may refer to a body to which the consumable assembly couples or connects to form an aerosol generation apparatus. Whilst the remains of the consumable assembly may be disposable or recyclable once the precursor has been consumed or the consumable assembly otherwise depleted, the consumer unit is reusable and configured to produce an aerosol from repeated couplings with consumable assemblies. For instance, the consumer unit may include a power supply to power the aerosol generation unit as well as electrical circuitry to control the aerosol generation process. The power supply may be rechargeable.

As used herein the term “E-liquid system” or “liquid system” may refer to the heating of a liquid or gel precursor in an airflow. As used herein the term “heat not burn” or “heated precursor” may refer to the heating of a precursor, typically tobacco, without combustion, or without substantial combustion (i.e. localised combustion may be experienced of limited portions of the precursor, including of less than 5% of the total volume).

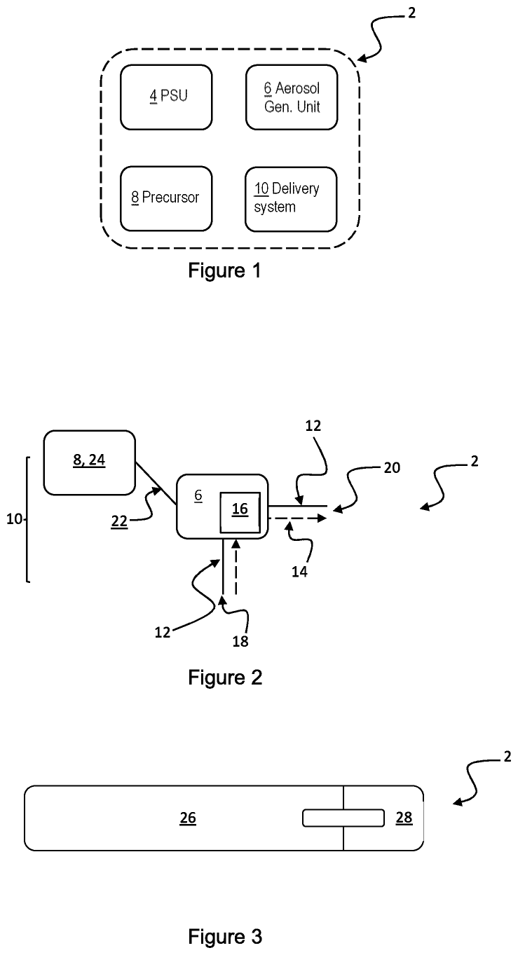

Referring to FIG. 1, an embodiment aerosol generating apparatus 2 includes a power supply 4, for supply of electrical energy. The apparatus 2 includes an aerosol generating unit 6 that is driven by the power supply 4. The power supply 4 may include an electric power supply in the form of a battery and/or an electrical connection to an external power source. The apparatus includes precursor 8, which in use is aerosolised by the aerosol generating unit 6. The apparatus 2 includes a delivery system 10 for delivery of aerosolised precursor to a user (not shown in FIG. 1).

Electrical circuitry (not illustrated in FIG. 1) may be implemented to control the interoperability of the power supply 4 and aerosol generating unit 6.

In variant embodiments, which are not illustrated, the power supply may be omitted, e.g. an aerosol generating unit implemented as an atomiser with flow expansion may not require a power supply.

Referring to FIG. 2, the aerosol generating apparatus 2 is an implementation of the embodiment of FIG. 1 and/or other embodiments disclosed herein, typically for generation of an aerosol from a liquid precursor. The delivery system 10 includes a flow path 12 that transmits flow 14 in operative proximity of a heating system 16 of the aerosol generating unit 6. By operative proximity it is meant that the flow is transmitted to carry vaporised and/or aerosol precursor generated from aerosol generating unit 6 to the outlet of the delivery system 10. The flow path 12 includes an inlet 18, and an outlet 20, which may be arranged as a mouthpiece. The delivery system 10 includes a precursor transmission system 22 to transmit the precursor 8, typically in liquid form from a storage 24, to the aerosol generating unit 6. The precursor transmission system 22 may be implemented as a wick, injector or other suitable device. In such embodiments, the precursor can be contained in a reservoir (not illustrated in FIG. 2) or other storage portion.

In variant embodiments, which are not illustrated, the precursor transmission system may be implemented by the aerosol generating unit, for example, as a porous heating component.

Referring to FIG. 3, which is a specific implementation of the embodiment of FIG. 2, a consumable assembly is implemented as a capsule/pod 28. The capsule 28 is a separate article to and connectable with a body 26 of a consumer unit. The consumer unit comprises the power supply 4. For instance the body houses the power supply 4. The capsule 28 includes the precursor 8, aerosol generating unit 6 and mouthpiece 20. A storage portion of the capsule 28, which is implemented as a reservoir (not shown in FIG. 3) is arranged to carry the precursor. In exemplary embodiments the capsule 28 therefore comprises a housing. Here, the housing defines or carries the storage portion.

As shown in FIG. 3, the housing of the consumable assembly, in the form of a capsule, comprises the precursor 8 as well as the aerosol generating unit 6 and the mouthpiece 20. However, in variant embodiments, which are not illustrated, one or both of the aerosol generating unit 6 and mouthpiece 20, are arranged as part of the consumer unit, e.g. the mouthpiece 20 is arranged as part of the body 26 and the precursor 6 and aerosol generating unit 6 are arranged as a separable cartomizer.

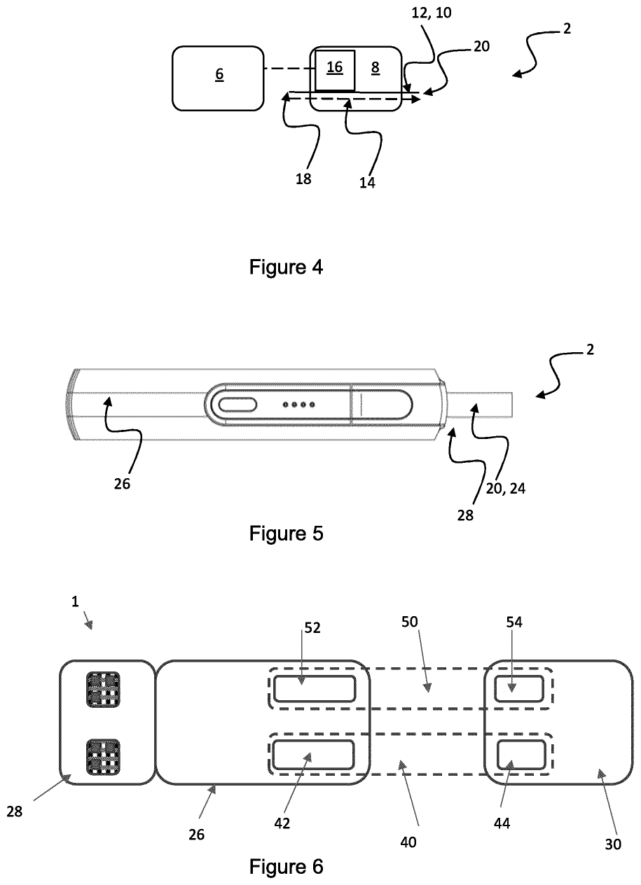

Referring to FIG. 4, the aerosol generating apparatus 2 is an implementation of the embodiment of FIG. 1 and/or other embodiments disclosed herein typically for generation of an aerosol from a solid precursor. A heating system 16 of the aerosol generating unit 6 interacts with the precursor 8 to generate vaporised and/or aerosol precursor. The precursor 8 is typically arranged as a solid and is arranged to receive thermal energy via conductive heat transfer from the aerosol generating unit 6, e.g. the heating system is arranged as a rod, which is inserted into the precursor or as a flat plate to abut a surface of the precursor. The delivery system 10 includes a flow path 12 that transmits flow 14 from an inlet 18 through (or in operative proximity to) the precursor 8 to carry the vapour and/or aerosol to an outlet 20 of the flow path 12.

Referring to FIG. 5, which is a specific implementation of the embodiment of FIG. 4, a consumable assembly is implemented as a stick 28. The stick 28 is separate to and connectable to a body 26 of a consumer device. The consumer device comprises the power supply 4 and aerosol generating unit 6. For instance the body houses the power supply 4 and aerosol generating unit 6. The stick 28 includes proximal the body 26 the precursor 8 (not shown in FIG. 5) as a reconstituted tobacco formulation and distal the body 26 a mouthpiece 20 arranged as a filter. A storage portion 24, which is implemented as wrapping material is arranged to carry the precursor 8.

Referring to FIG. 6 an aerosol generating system 1, which may be implemented in any of the preceding embodiments, comprises an aerosol generation apparatus 2 and an external device 30. The aerosol generation apparatus comprises a consumable assembly 28 and a consumer unit 26. Here, the consumable assembly 28 is operatively connectable to the consumer unit to form the aerosol generation apparatus and to be used by a user to generate an aerosol as herein described. For instance, the consumable assembly may be a stick for a HNB process, but the exemplary embodiment will be herein described in relation to a pod consumable containing a liquid precursor.

The aerosol generating system 1 comprises a processor 40. The processor 40 controls the operation of the aerosol generating system 1. For instance, the processor controls the aerosol generation process. In the exemplary embodiment, the processor also controls a communication interface 50. The communication interface establishes a communication link between the consumer unit 26 and the external device 30. As described above, the external device 30 may be part of a network, but will be herein described as being a portable device such as mobile phone. It will be appreciated that the mobile phone may also be connected to a network, for instance to access a remotely stored reference table or other lookup information. That is the operation of the mobile phone, as an exemplary external device, may be distributed across a network.

The processor 40 is shown as comprising a processor unit 42 on the consumer unit 26 (a consumer processor unit) and a processor 44 on the external device (a external processor unit). That is the function of the processor may be distributed across the consumer unit and the external device. For instance, so that the consumer unit can operate to generate an aerosol without requiring a continuous communication link to be established by the communication interface.

The communication interface 50 is shown as comprising a first communication module 52 on the consumer unit and a second communication module on the external device. The respective first and second communication modules establishing a known communication link, for instance, suitably a wireless communication link such as a near field communication link. Here, the communication link may operate a known communication and pairing protocol such as Wi-Fi or Bluetooth pairing.

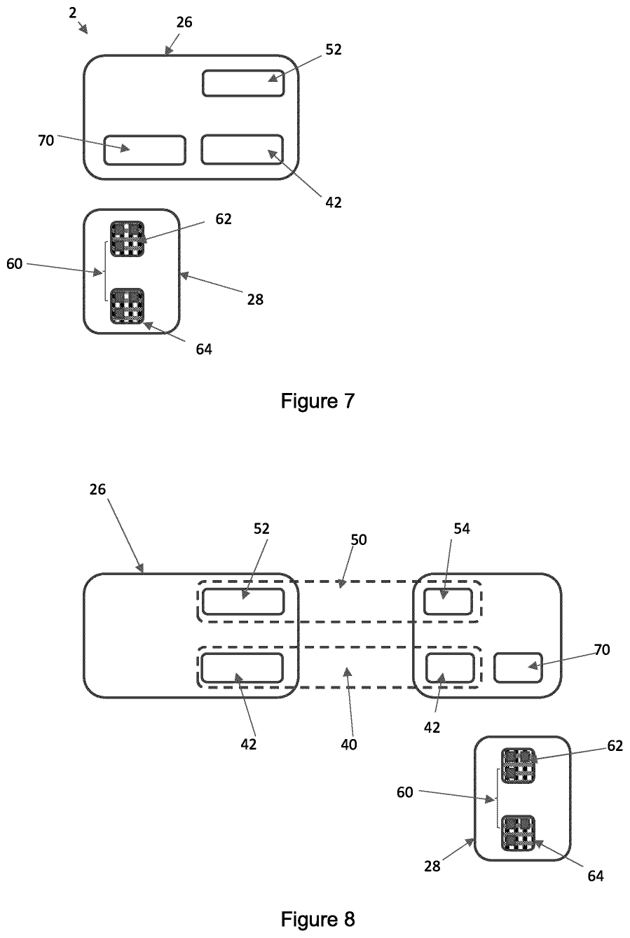

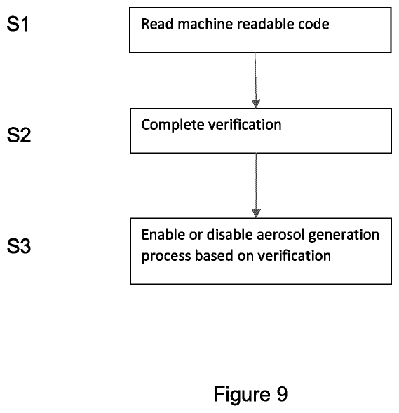

Referring to FIG. 7, an exemplary aerosol generating apparatus 2 is shown. The consumable assembly 28 is shown as including a machine readable code 60 and the consumer unit 28 a machine 70 for reading the machine readable code 60. In use, before operatively coupling the consumable 28 with the consumer unit 26, the machine readable code 60 is first read by the machine 70. As shown in FIG. 9, using the machine 70 to read the machine readable code 60 completes the step S1. In some suitable embodiments, the processor 40 is configured to prompt the user to complete the machine reading step. For instance, the processor 40 may be configured to provide an alert or other indicator. In some embodiments, the processor is configured to prompt a machine reading step each after a consumable is removed from operative engagement with the consumer unit. The prompt may comprise the processor being configured to disable the consumer unit from being operated to cause an aerosol until the machine readable step has been completed. The processor 40 may be configured to disable the consumer unit by controlling a power supply of the consumer unit not to provide power to an aerosol generation unit or the like.

When the machine 70 reads the machine readable code 60, the processor 40 is configured to compete a verification step. As shown in FIG. 9, the verification step is shown as step S2.

In one exemplary embodiment, the verification step authenticates the consumable as an authorised consumable 28. For instance, the verification step comprises deriving from the read code a unique reference for the consumable 28. The unique reference is then looked up in a reference table, for instance, a reference table may be hosted by a network or other external device. Here the verification step may comprise verifying the consumable as an authorised consumable if the read unique reference is present in the reference table. In the event the unique reference is not found on the reference table, the consumable can be determined to be unauthorised. Alternatively, the reference table may include data related to the unique reference. As described below, the verification step may comprise comparing the data related to the unique reference against other data connected to the user or external device. Furthermore, the data related to the unique reference may be updated. Thus the reference table may be configured to track the consumable 28 through the distribution and sales process. Here, if a step in the distribution and sales process is not completed, an otherwise genuine consumable (i.e. not counterfeited) can still be determined to be unauthorised. For instance, the reference table can be updated by manufacturers or retailer to show that a distribution step or delivery step or sales step has been completed and if it has not the consumable may be determined as unauthorised. By way of example, if a consumable becomes lost or stolen are manufacture, a sales step by a retailer would not be completed and the genuine consumable can be verified as unauthorised, because the data related to the unique reference would not be correct in the reference table.

In one exemplary embodiment, the verification step comprises completing an age verification step. For instance, the external device, suitably a mobile phone, is configured to run an application to control the consumer unit as is known in the art. Here, the application may be part of the processor. As part of operating the application on the mobile phone, the user may be required to complete an age validation step. Here, the verification step may comprise deriving from the read code an age limit associated with the consumable and verifying the consumable as authenticated if the user's age determined in the age validation step is greater than the age limit. Alternatively, the age data related to the consumable may be contained in the reference table associated with a unique reference of the consumable. By including an age verification step in the verification process, the age of the user can be validated each time a consumable is paired with the consumer unit, wherein the age verification requires the consumer unit to be in proximity to the external device which may be assumed to be carried by the user whose age has been verified.

In one exemplary embodiment, the verification step comprises a location verification. Here, the data read from the machine readable code 60 may pertain to a geographic area of use or the data read pertaining to the consumable may be a unique reference and the geographic area data may be contained in a reference table that can be looked up using the unique reference. In the exemplary embodiments, the location data pertaining to the consumable can be referenced against a location of the consumer unit and the consumable validated as an authorised consumable or determined to be an authorised consumable based on whether the location of the consumer unit is allowed. In the exemplary embodiments comprising an aerosol generating system wherein the consumer unit is in communication with an external device such as a portable device like a mobile phone, the location of the external device may be used as an estimate of the location of the consumer unit.

If the consumable is verified in the verification step S2, the processor can control the consumer unit to complete an aerosol generation process on user demand under step S3 of FIG. 9. That is, the consumer unit can be enabled to function normally. If the consumable is verified as an unauthorised consumable in the verification step S2, the consumer unit can be disabled in step S3 so that the consumer unit cannot be operated to complete a usual aerosol generation process. That is, the consumer unit is disabled. The consumer unit can remain disabled until a further consumable is provided for verification. Alternatively, the user may complete a further step to authorise the consumable. For instance by contacting a seller or otherwise taking action to have, for instance, the data in the reference table properly updated so that a further reading by the machine of the machine readable code, results in a verification of the consumable.

Referring to FIG. 7, an aerosol generating system 1 is shown wherein the machine 70 for reading the machine readable code 60 is provided on an external device 30. For instance, the system may make use of the operability of the external device, rather than repeat the hardware on the consumer unit. As explained below, in some exemplary embodiments, the machine readable code is an optical code. Here the machine 70 can be a camera, and the on-board camera of a paired mobile phone can therefore be used to read the code, rather than provide a camera on the consumer unit. Likewise, if the machine readable code is an electronic antenna, for instance a RFID antenna, the readable capability of the mobile phone (as an exemplary external device) can be utilised.

In exemplary embodiments, the machine readable code 60 is an optical code. Here, the machine for reading the optical code is an optical sensor, for instance a camera 70. The optical code is provided on an outer surface of the consumable assembly. For instance, the optical code may be etched or otherwise affixed to the outer surface. In a pod consumable, the outer surface may be an outer surface of a tank or an outer surface of an end of the consumable assembly. In a HNB stick consumable, the outer surface may be an external wrapping of external filter surface. Suitably, the outer surface may be an end of the consumable. The end may be opposed to the end that is operatively engaged. That is, the optical code may be provided on an end of the consumable assembly that is not inserted or otherwise engaged with the consumer device.

The optical code 60 is shown suitably as being a distributed optical code 60. Here, the optical code includes a plurality of discrete code-portions provided at respective positions on said surface, and said data is distributed across said code-portions. As shown in FIGS. 7 and 8, the optical code 60 is suitably shown as a first discrete code portion 62 and a second discrete code portion 64. But it will be appreciated that, third, or fourth or further code portions can be added as required or suitable. The first and second discrete code portions may be separated on the packaging. For instance in a line or other arrangement. Each discrete code portion may be a QR code as is known in the art. Because the data is distributed across the discrete code portion, reading one code portion will not enable information pertaining to the consumable and, for instance, necessary to complete a verification step, to be derived. The distributed code can be combined or stitched back together in software once all the discrete code portions have been read. Distributing the data across a plurality of discrete code portions can assist in defeating counterfeiters, as well as improving the ability to fit the code on the consumable, and also requiring software to be adapted to be able to read the distributed code. Thus, if the aerosol generating apparatus is paired with an external device it can be harder to avoid running a dedicated application to read the consumable and complete the verifications step for instance by running alternative applications that bypass the intended verification process.

There is therefore provided an improved consumable, aerosol generating apparatus, and aerosol generating system wherein the consumable can be tracked through the manufacturing and/or sales process and verified based on one or more of the steps having been completed. Moreover, by distributing the data pertaining to the consumable across two or more of a plurality of discrete optical codes, the process is improved by reducing the ability to subvert verification steps using non-authorised verification platforms. Also, by completing a verification process each time a consumable is inserted or operatively coupled to a consumer device, the verification can require an age validation with an external device that is generally carried by the user whose age has been verified.

1. An aerosol generating apparatus comprising:
a consumable, the consumable comprising;
an aerosol forming precursor; and
data pertaining to the consumable encoded in machine-readable code provided on a surface of the consumable,
wherein
said machine-readable code comprises a plurality of discrete optical code-portions provided at spaced locations on said surface, and said data is distributed across said code-portions;
a consumer unit configured to operatively engage with the consumable;
a machine for reading said machine-readable code; and
a processor configured to determine the data pertaining to the consumable by combining information that is distributed across two or more of the discrete optical code-portions.

2. The aerosol generating apparatus of claim 1, wherein each discrete optical code-portion comprises a respective QR code.

3. (canceled)

4. The aerosol generating apparatus of claim 1, wherein:
the processor is configured to control the consumer unit to enable or disable an aerosol generation process based on a verification process;
wherein the processor is configured to complete the verification process by comparing the determined data pertaining to the consumable to a reference.

5. The aerosol generating apparatus of claim 4, wherein the processor is configured to disable the consumer unit by controlling a power supply of the consumer unit to not provide power to the aerosol generating apparatus.

6. The aerosol generating apparatus of claim 1, wherein the machine for reading the machine-readable code is a camera housed on the consumer unit.

7. The aerosol generating apparatus of claim 1, further comprising an external device in communication with the consumer unit, wherein the machine for reading the machine-readable code is a camera housed on the external device.

8. The aerosol generating apparatus of claim 7, wherein the processor is distributed across the consumer unit and the external device.

9. A method of verifying a consumable in an aerosol generating apparatus, wherein the consumable comprises data pertaining to the consumable encoded in machine-readable code provided on a surface of the consumable, wherein said machine-readable code comprises a plurality of discrete optical code-portions provided at spaced locations on said surface, and said data is distributed across said code-portions, wherein the method comprises the steps of:
reading a first of said discrete optical code portions provided on said consumable;
reading a second of said discrete optical code portions provided on said consumable;
deriving from the read first and second discrete optical code portions the data pertaining to the consumable;
completing a verification step by comparing the data pertaining to the consumable against a reference.

10. The method of claim 9, wherein the verification step is an age verification step and the method comprises deriving data from the consumable pertaining to an authorised user age and the reference is a validated user age associated with a validation step completed by the user.

11. The method of claim 9, wherein the verification step is a location verification step and the method comprises deriving data from the consumable pertaining to an authorised geographical area of the use of the consumable and the reference is a geographical location of the consumer unit.

12. The method of claim 9, wherein the verification step is a verification step configured to authenticate the consumable as an authorised consumable and the method comprises deriving unique data from the consumable and the reference comprises a list of authorised consumables.

13. The method of claim 12, wherein the unique data derived from reading the machine-readable code is a unique reference to identify the consumable.

14. The method of claim 13, wherein the method comprises updating the reference comprising the list of authorised consumables with data associated to the unique reference based on one or more steps being completed in the manufacturing or sales process, and determining whether the consumable is authorized based on the updated reference.

15. A computer readable medium executable to carry out a method of verifying a consumable in an aerosol generating apparatus, wherein the consumable comprises data pertaining to the consumable encoded in machine-readable code provided on a surface of the consumable, wherein said machine-readable code comprises a plurality of discrete optical code-portions provided at spaced locations on said surface, and said data is distributed across said code-portions, wherein the method comprises the steps of:
reading a first of said discrete optical code portions provided on said consumable;
reading a second of said discrete optical code portions provided on said consumable;
deriving from the read first and second discrete optical code portions the data pertaining to the consumable;
completing a verification step by comparing the data pertaining to the consumable against a reference.
